(19) United States
(12) Patent Application Publication
(10) Pub. No.: US 2010/0233082 A1
(43) Pub. Date: Sep. 16, 2010

(54) 68GA-Labeled Peptide-Based Radiopharmaceuticals

(76) Inventors: Bengt Langstrom, Uppsala (SE); Irina Velikyan, Uppsala (SE); Mats Bergstrom, London (GB); Orjan Lindhe, Belinge (SE)

Correspondence Address: GE HEALTHCARE, INC., IP DEPARTMENT 101 CARNEGIE CENTER, PRINCETON, NJ, 08540-6231, US

(21) Appl. No.: 12/376,800

(22) PCT Filed: Aug. 28, 2007
(86) PCT No.: PCT/IB07/02479

§ 371 Date: May 24, 2010

Related U.S. Application Data
(60) Provisional application No. 60/823,682, filed on Aug. 28, 2006.

(51) Int. Cl. A61K 51/08; C07K 7/50

(52) U.S. Cl. 424/1.69; 530/328; 530/327; 530/326

(57) ABSTRACT

The invention relates to new peptide-based compounds for use as diagnostic imaging agents or as therapeutic agents wherein the agents comprise targeting vectors which bind to integrin receptors.

### FIELD OF THE INVENTION

The present invention relates to new radiolabelled peptide-based compounds and their use in diagnostic imaging, pretherapeutical dosimetry, therapy planning, therapy monitoring or radiotherapy. More specifically the invention relates to the use of such peptide-based compounds as targeting vectors that bind to receptors associated with angiogenesis, in particular integrin receptors, e.g. the αᵥβ₃ integrin receptor. Such imaging agents may thus be used for diagnosis of for example malignant diseases, heart diseases, endometriosis, inflammation-related diseases, rheumatoid arthritis and Kaposi's sarcoma. Moreover such agents may be used in pretherapeutical dosimetry, therapy planning and therapy monitoring of these diseases.

### BACKGROUND OF THE INVENTION

New blood vessels can be formed by two different mechanisms: vasculogenesis or angiogenesis. Angiogenesis is the formation of new blood vessels by branching from existing vessels. The primary stimulus for this process may be inadequate supply of nutrients and oxygen (hypoxia) to cells in a tissue. The cells may respond by secreting angiogenic factors, of which there are many; one example, which is frequently referred to, is vascular endothelial growth factor (VEGF). These factors initiate the secretion of proteolytic enzymes that break down the proteins of the basement membrane, as well as inhibitors that limit the action of these potentially harmful enzymes. The other prominent effect of angiogenic factors is to cause endothelial cells to migrate and divide. Endothelial cells that are attached to the basement membrane, which forms a continuous sheet around blood vessels on the contralumenal side, do not undergo mitosis. The combined effect of loss of attachment and signals from the receptors for angiogenic factors is to cause the endothelial cells to move, multiply, and rearrange themselves, and finally to synthesise a basement membrane around the new vessels.

Angiogenesis is prominent in the growth and remodelling of tissues, including wound healing and inflammatory processes. Tumors must initiate angiogenesis when they reach millimetre size in order to keep up their rate of growth. Angiogenesis is accompanied by characteristic changes in endothelial cells and their environment. The surface of these cells is remodelled in preparation for migration, and cryptic structures are exposed where the basement membrane is degraded, in addition to the variety of proteins which are involved in effecting and controlling proteolysis. In the case of tumours, the resulting network of blood vessels is usually disorganised, with the formation of sharp kinks and also arteriovenous shunts. Inhibition of angiogenesis is also considered to be a promising strategy for antitumour therapy. The transformations accompanying angiogenesis are also very promising for diagnosis, an obvious example being malignant disease, but the concept also shows great promise in inflammation and a variety of inflammation-related diseases, including atherosclerosis, the macrophages of early atherosclerotic lesions being potential sources of angiogenic factors. These factors are also involved in re-vascularisation of infarcted parts of the myocardium, which occurs if a stenosis is released within a short time.

Further examples of undesired conditions that are associated with neovascularization or angiogenesis, the development or proliferation of new blood vessels are shown below. Reference is also made in this regard to WO 98/47541.

Diseases and indications associated with angiogenesis are e.g. different forms of cancer and metastasis, e.g. breast, skin, colorectal, pancreatic, prostate, lung or ovarian cancer.

Other diseases and indications are inflammation (e.g. chronic), atherosclerosis, rheumatoid arthritis and gingivitis.

Further diseases and indications associated with angiogenesis are arteriovenous alformations, astrocytomas, choriocarcinomas, glioblastomas, gliomas, hemangiomas (childhood, capillary), hepatomas, hyperplastic endometrium, ischemic myocardium, endometriosis, Kaposi sarcoma, macular degeneration, melanoma, neuroblastomas, occluding peripheral artery disease, osteoarthritis, psoriasis, retinopathy (diabetic, proliferative), scleroderma, seminomas and ulcerative colitis.

Angiogenesis involves receptors that are unique to endothelial cells and surrounding tissues. These markers include growth factor receptors such as VEGF and the Integrin family of receptors. Immunohistochemical studies have demonstrated that a variety of integrins perhaps most importantly the class are expressed on the apical surface of blood vessels [Conforti, G., et al. (1992) Blood 80: 37-446] and are available for targeting by circulating ligands [Pasqualini, R., et al. (1997) Nature Biotechnology 15: 542-546]. The α5β1 is also an important integrin in promoting the assembly of fibronectin matrix and initiating cell attachment to fibronectin. It also plays a crucial role in cell migration [Bauer, J. S., (1992) J. Cell Biol. 116: 477-487] as well as tumour invasion and metastasis [Gehlsen, K. R., (1988) J. Cell Biol. 106: 925-930].

The integrin αᵥβ₃ is one of the receptors that is known to be associated with angiogenesis. Stimulated endothelial cells appear to rely on this receptor for survival during a critical period of the angiogeneic process, as antagonists of the αᵥβ₃ integrin receptor/ligand interaction induce apoptosis and inhibit blood vessel growth.

Integrins are heterodimeric molecules in which the α- and β-subunits penetrate the cell-membrane lipid bilayer. The α-subunit has four Ca²⁺ binding domains on its extracellular chain, and the β-subunit has a number of extracellular cysteine-rich domains.

Many ligands (eg. fibronectin) involved in cell adhesion contain the tripeptide sequence arginine-glycine-aspartic acid (RGD). The RGD sequence appears to act as a primary recognition site between the ligands presenting this sequence and receptors on the surface of cells. It is generally believed that secondary interactions between the ligand and receptor enhance the specificity of the interaction. These secondary interactions might take place between moieties of the ligand and receptor that are immediately adjacent to the RGD sequence or at sites that are distant from the RGD sequence.

RGD peptides are known to bind to a range of integrin receptors and have the potential to regulate a number of cellular events of significant application in the clinical setting. (Ruoslahti, J. Clin. Invest., 87: 1-5 (1991)). Perhaps the most widely studied effect of RGD peptides and mimetics thereof relate to their use as anti-thrombotic agents where they target the platelet integrin GpIIbIIIa.

Inhibition of angiogenesis in tissues by administration of either an αvβ3 or αvβ5 antagonist has been described in for example WO 97/06791 and WO 95/25543 using either antibodies or RGD containing peptides. EP 578083 describes a series of monocyclic RGD containing peptides and WO 90/14103 claims RGD-antibodies. Haubner et al. in the J. Nucl. Med. (1999); 40: 1061-1071 describe a new class of tracers for tumour targeting based on monocyclic RGD containing peptides. Biodistribution studies using whole-body autoradiographic imaging revealed however that the ¹²⁵I-labelled peptides had very fast blood clearance rates and predominantly hepatobiliary excretion routes resulting in high background.

Cyclic RGD peptides containing multiple bridges have also been described in WO 98/54347 and WO 95/14714. Peptides derived from in vivo biopanning (WO 97/10507) have been used for a variety of targeting applications. The sequence CDCRGDCFC (RGD-4C), has been used to target drugs such as doxirubicin (WO 98/10795), nucleic acids and adenoviruses to cells (see WO 99/40214, WO 99/39734, WO 98/54347, WO 98/54346, U.S. Pat. No. 5,846,782). Peptides containing multiple cysteine residues do however suffer from the disadvantage that multiple disulphide isomers can occur. A peptide with 4 cysteine residues such as RGD-4C has the possibility of forming 3 different disulphide folded forms. The isomers will have varying affinity for the integrin receptor as the RGD pharmacophore is forced into 3 different conformations.

Further examples of RGD comprising peptide-based compounds are found in PCT/NO01/00146 and PCT/NO01/00390, the content of which are incorporated herein by reference.

The efficient targeting and imaging of integrin receptors associated with angiogenesis in vivo demands therefore a selective, high affinity RGD based vector that is chemically robust and stable. Furthermore, the route of excretion is an important factor when designing imaging agents in order to reduce problems with background. These stringent conditions are met by the bicyclic structures described in the present invention.

⁶⁸Ga-based peptide tracers offer a superior vehicle for tumor imaging/diagnosis, chemo- and radiotherapy planning and monitoring as well as pretherapeutical dosimetry for radiotherapy. Furthermore, after the diagnosis and dosimetry ⁶⁸Ga might be substituted in the same vector with a therapeutical radionuclide (⁸⁷Y₃₉, ²¹³Bi₈₃, ¹⁷⁷Lu₇₁) for subsequent radiotherapy. A straightforward preparation of a tracer using radiometallation with generator produced ⁶⁸Ga may result in kit type production of PET radiopharmaceuticals and make PET examinations possible at centres lacking accelerators.

### SUMMARY OF THE INVENTION

In one aspect, the invention provides new ⁶⁸Ga peptide-based compound of Formula I as defined in the claims. These compounds have affinity for integrin receptors, e.g. affinity for the integrin αᵥβ₃.

The present invention also provides a pharmaceutical composition comprising an effective amount for diagnostic imaging (e.g. an amount effective for enhancing image results in in vivo imaging) of a compound of general formula I or a salt thereof, together with one or more pharmaceutically acceptable adjuvants, excipients or diluents.

The invention further provides a pharmaceutical composition for pretherapeutical dosimetry, therapy planning and therapy monitoring of a disease comprising an effective amount of a compound of general formula I, or an acid addition salt thereof, together with one or more pharmaceutically acceptable adjuvants, excipients or diluents.

A positron emitting radiometal attached to a chelating agent on a peptide ligand such as compound of formula I can be used in pretherapeutical dosimetry. This would provide information on how much toxic radioactivity goes to the tumor and if normal organs are affected. It is an easy and quick estimation of dose to tumor and normal tissue.

The PET-radiopharmaceuticals of formula I can also be used for chemo- and radiotherapy planning. The PET-tracer uptake by a certain receptor over expressing tissue can be quantified to provide a measure of receptor density in vivo. Based on the receptor density value an appropriate therapy type may be subscribed or advised against.

Viewed from a further aspect the invention provides the use of a compound of formula I for the manufacture of a diagnostic imaging agent, preferably a PET tracer, for use in a method of diagnosis involving administration of said diagnostic imaging agent to a human or animal body and generation of an image of at least part of said body.

Viewed from a still further aspect the invention provides a method of generating an image of a human or animal body involving administering a diagnostic imaging agent to said body, e.g. into the vascular system and generating an image of at least a part of said body to which said diagnostic imaging agent has distributed using scintigraphy, PET or SPECT modalities, wherein as said diagnostic imaging agent is used an agent of formula I.

Viewed from a further aspect the invention provides method of monitoring the effect of treatment of a human or animal body with a drug to combat a condition associated with cancer, preferably angiogenesis, e.g. a cytotoxic agent or radiotherapeutics said method involving administering to said body a compound of formula I, or an acid addition salt thereof, together with one or more pharmaceutically acceptable adjuvants, excipients or diluents, and detecting the uptake of said agent by cell receptors, preferably endothelial cell receptors and in particular αᵥβ₃ receptors, said administration and detection optionally but preferably being effected repeatedly, e.g. before, during and after treatment with said drug.

Further, it provides a method of pretherapeutical dosimetry to measure the radioactivity amount taken up by tumors and normal organs. This would provide information on how much toxic radioactivity goes to the tumor and if normal organs are affected. It is an easy and quick estimation of dose to tumor and normal tissue. The said method involves administering to said body an agent of formula I and detecting the uptake of said agent by cell receptors, preferably endothelial cell receptors and in particular αᵥβ₃ receptors, said administration and detection being conducted before the treatment and for planning the treatment.

Still further, it provides a method of therapy planning to determine the appropriate therapy type. This would provide information of receptor density in vivo. The said method involves administering to a human or animal body an agent of formula I, or an acid addition salt thereof, together with one or more pharmaceutically acceptable adjuvants, excipients or diluents, quantifying the receptor density in vivo by measuring receptor uptake over expressing tissue and determining appropriate therapy type.

Finally, it provides compounds of Formula III, IV and V and methods of using compounds III, IV and V for radiotherapy of cancer, preferably angiogenesis. It also provides a method of radiotherapy of cancer, preferably angiogenesis comprises administering to a human or animal body an effective amount of agent of compounds of Formula III, IV or V. It further provides the use of such a compound for the manufacture of a medicament for the radiotherapy treatment in a human or animal.

### DETAILED DESCRIPTION OF THE INVENTION

Viewed from one aspect the invention provides new peptide-based compounds of Formula I as defined in the claims. These compounds have affinity for integrin receptors, e.g. affinity for the integrin αvβ3.

The compounds of Formula I comprise at least two bridges, wherein one bridge forms a disulphide bond and the second bridge comprises a thioether (sulphide) bond and wherein the bridges fold the peptide moiety into a ‘nested’ configuration.

The compounds of the current invention thus have a maximum of one disulphide bridge per molecule moiety. Compounds defined by the present invention are surprisingly stable in vivo.

These new compounds may be used in diagnostic imaging as well as for pretherapeutical dosimetry, therapy planning and therapy monitoring. The new peptide-based compounds described in the present invention are defined by Formula I:

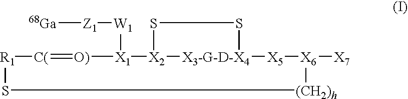

or physiologically acceptable salts thereof

wherein
G represents glycine, and
D represents aspartic acid, and
R₁ represents —(CH₂)ₙ— or —(CH₂)ₙ—C₆H₄—, preferably R₁ represents —(CH₂)—, and
n represents a positive integer between 1 and 10, and
h represents a positive integer 1 or 2, and
X₁ represents an amino acid residue wherein said amino acid possesses a functional side-chain such as an acid or amine preferentially aspartic or glutamic acid, lysine, homolysine, diaminoalkylic acid or diaminopropionic acid,
X₂ and X₄ represent independently an amino acid residue capable of forming a disulphide bond, preferably a cysteine or a homocysteine residue, and
X₃ represents arginine, N-methylarginine or an arginine mimetic, preferably an arginine, and
X₅ represents a hydrophobic amino acid or derivatives thereof, preferably a tyrosine, a phenylalanine, a 3-iodo-tyrosine or a naphthylalanine residue, and more preferably a phenylalanine or a 3-iodo-tyrosine residue, and
X₆ represents a thiol-containing amino acid residue, preferably a cysteine or a homocysteine residue, and
X₇ is absent or represents a homogeneous biomodifier moiety preferably based on a monodisperse PEG building block comprising 1 to 10 units of said building block, said biomodifier having the function of modifying the pharmacokinetics and blood clearance rates of the said agents. In addition X₇ may also represent 1 to 10 amino acid residues preferably glycine, lysine, aspartic acid or serine. In a preferred embodiment of this invention X₇ represents a biomodifier unit comprised of polymerisation of the monodisperse PEG-like structure, 17-amino-5-oxo-6-aza-3,9,12,15-tetraoxaheptadecanoic acid of Formula II,

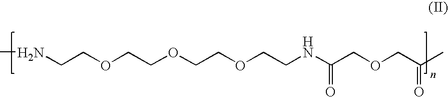

wherein n equals an integer from 1 to 10 and where the C-terminal unit is an amide moiety.
W₁ is absent or represents a spacer moiety and is preferentially derived from glutaric and/or succinic acid and/or a polyethyleneglycol based unit and/or a unit of Formula II

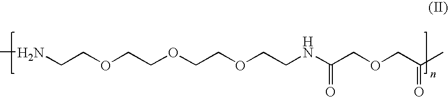

Z₁ is chelating agents of formulas given in Table I.

Conjugates comprising chelating agents can be radiolabelled to give good radiochemical purity, RCP, at room temperature, under aqueous conditions at near neutral pH. The risk of opening the disulphide bridges of the peptide component at room temperature is less than at an elevated temperature. A further advantage of radiolabelling the conjugates at room temperature is a simplified procedure in a hospital pharmacy.

The role of the spacer moiety W₁ is to distance the relatively bulky chelating agent from the active site of the peptide component. The spacer moiety W₁ is also applicable to distance a bulky antineoplastic agent from the active site of the peptide.

It is found that the biomodifier, X₇, modifies the pharmacokinetics and blood clearance rates of the compounds. The biomodifier effects less uptake of the compounds in tissue i.e. muscle, liver etc. thus giving a better diagnostic image due to less background interference. The secretion is mainly through the kidneys due to a further advantage of the biomodifier.

Compounds defined in Formula I also comprises chelating agents, Z1, as defined in Table I.

TABLE 1

| Class | | |
|---|---|---|
| of | | |
| ligand | Structure | Definitions |
| MAG3 type | 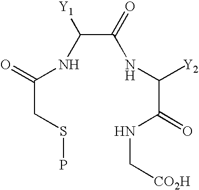 | P = protecting group (preferably. benzoyl, acetyl, EOE); Y1, Y2 contains a suitable functionality such that it can be conjugated to the peptide vector; preferably H (MAG3), or the side chain of any amino acid, in either L or D form. |
| G4 type ligands | 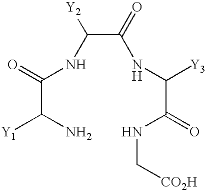 | Y1, Y2, Y3-contains a suitable functionality such that it can be conjugated to the peptide vector; preferably H, or the side chain of any amino acid, in either L or D form. |
| Tetra-amine ligands | 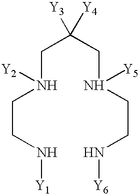 | Y1-Y6 can be H, alkyl, aryl or combinations thereof where the Y1-6 groups contain one or more functional moieties such that the chelate can be conjugated to the vector-e.g. preferably alkylamine, alkylsulphide, alkoxy, alkyl carboxylate, arylamine, aryl sulphide or α-haloacetyl |
| Macrocyclic ligands such as 1,4,7- triazacyclo- nonanetriacetic acid (NOTA), 1,4,7,10- tetraazacyclo dodecanetetra acetic acid (DOTA), 1,4,8,11- tetraazacyclo- tetradecane- 1,4,8,11- tetraacetic acid (TETA) and their derivatives | 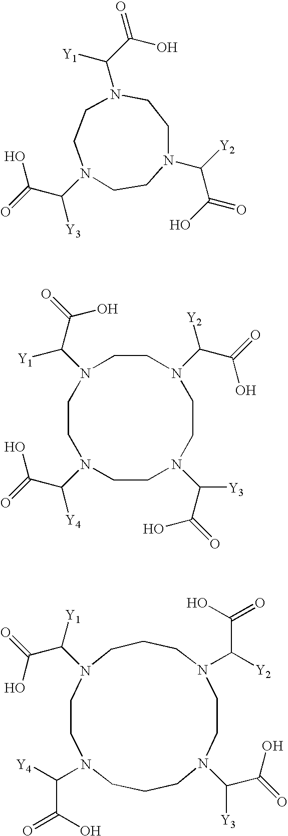 | Y1 can be H or contain one or more functional moieties such that the chelate can be conjugated to the vector-e.g. preferably alkylamine, alkylsulphide, alkoxy, alkyl carboxylate, arylamine, aryl sulphide or α-haloacetyl Y2-4 can be H or contain one or more functional moieties that would on the one hand improve the complexation depending on a particular metal cation and on the other hand change the overall charge and hydrophilicity of the complex in order to modify the pharmacokinetics and blood clearance rates e.g. alkylamine, alkoxy, alkyl carboxylate, phenol, hydroxamate, aryl sulphide, alkyl. |
| Cylam type ligands | 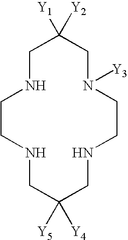 | Y1-5 can be H, alkiyl, aryl or combinations thereof and where Y1-5 groups contain one or more functional moieties such that the chelate can be conjugated to the vector-e.g. preferably alkylamine, alkylsulphide, alkoxy, alkyl carboxylate, arylamine, aryl sulphide or α-haloacetyl |
| Diamine- diphenol | 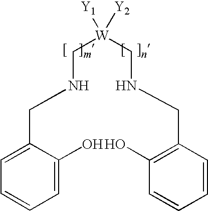 | Y1-Y2-H, alkyl, aryl and where Y1 or Y2 groups contains a functional moiety such that the chelate can be conjugated to the vector-e.g. preferably alkylamine, alkylsulphide, alkoxy, alkyl carboxylate, arylamine, aryl sulphide or α-haloacetyl W = C,N M′ = n′ = 1 or 2 |
| HYNIC | 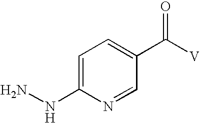 | V = linker to vector or vector itself. |
| Amide thiols | 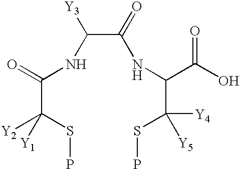 | P = protecting group (preferably. benzoyl, acetyl,EOE); Y 1-5 = H, alkyl, aryl; or Y3 is a L or D amino acid side- chain or glycine and the carboxylate may be used for conjugation to the vector via an amide bond. Alternatively the R₁₋₅ groups may contain additional functionality such that the chelate can be conjugated to the vector-e.g. alkylamine, alkylsulphide, alkoxy, alkyl carboxylate, arylamine, aryl sulphide or α-haloacetyl. |

The peptide component of the conjugates described herein have preferably no free amino- or carboxy-termini. This introduces into these compounds a significant increase in resistance against enzymatic degradation and as a result they have an increased in vivo stability as compared to many known free peptides.

As used herein the term ‘amino acid’ refers in its broadest sense to proteogenic L-amino acids, D-amino acids, chemically modified amino acids, N-methyl, Cα-methyl and amino acid side-chain mimetics and unnatural amino acids such as naphthylalanine. Any naturally occurring amino acid or mimetics of such natural occurring amino acids are preferred.

Some preferred embodiment of the compounds of formula I is illustrated by compound I below:

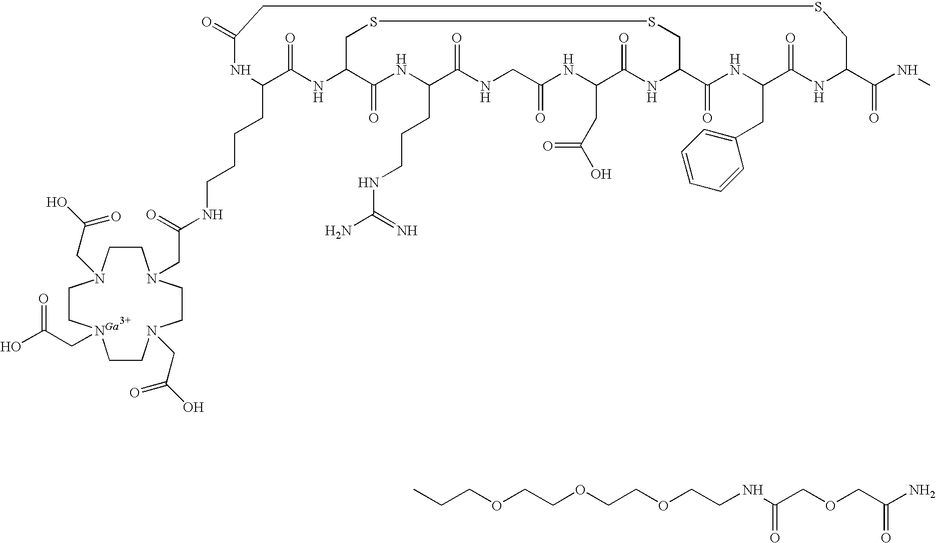

In most cases, it is preferred that the amino acids in the peptide are all in the L-form. However, in some embodiments of the invention one, two, three or more of the amino acids in the peptide are preferably in the D-form. The inclusion of such D-form amino acids can have a significant effect on the serum stability of the compound. According to the present invention, any of the amino acid residues as defined in formula I may preferably represent a naturally occurring amino acid and independently in any of the D or L conformations.

Some of the compounds of the invention are high affinity RGD based vectors. As used herein the term ‘high affinity RGD based vector’ refers to compounds that have a Ki of <10 nM and preferably <5 nM, in a competitive binding assay for αvβ3 integrin and where the Ki value was determined by competition with the known high affinity ligand echistatin. Methods for carrying out such competition assays are well known in the art.

The present invention also provides a pharmaceutical composition comprising an effective amount for diagnostic imaging (e.g. an amount effective for enhancing image contrast in in vivo imaging) of a compound of general formula I or a salt thereof, together with one or more pharmaceutically acceptable adjuvants, excipients or diluents.

The invention further provides a pharmaceutical composition for treatment of a disease comprising an effective amount of a compound of general formula III, IV and V, or an acid addition salt thereof, together with one or more pharmaceutically acceptable adjuvants, excipients or diluents.

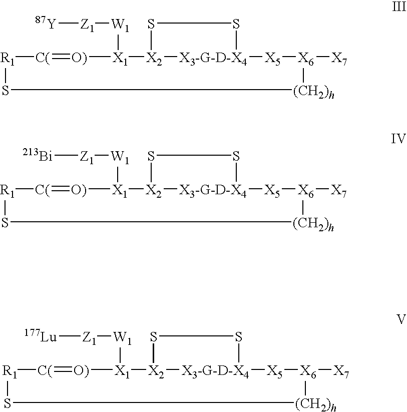

Other representative spacer (W₁) elements include structural-type polysaccharides, storage-type polysaccharides, polyamino acids and methyl and ethyl esters thereof, and polypeptides, oligosaccharides and oligonucleotides, which may or may not contain enzyme cleavage sites.

The chelating agents (Z₁) in the imaging agents of the invention may be any chelator capable of forming stable complexes with ⁶⁸Ga and/or therapeutical radionuclides ⁸⁷Y₃₉, ²¹³Bi₈₃, ¹⁷⁷_{Lu}₇₁.

The metal ions of the instant invention can be easily complexed to the chelating agent, for example, by merely exposing or mixing an aqueous solution of the chelating agent-containing moiety with a metal salt in an aqueous solution preferably having a pH in the range of about 4 to about 11. The salt can be any salt, but preferably the salt is a water soluble salt of the metal such as a halogen salt, and more preferably such salts are selected so as not to interfere with the binding of the metal ion with the chelating agent. The chelating agent-containing moiety is preferably in aqueous solution at a pH of between about 5 and about 9, more preferably between pH about 6 to about 8. The chelating agent-containing moiety can be mixed with buffer salts such as HEPES, citrate, carbonate, acetate, phosphate and borate to produce the optimum pH. Preferably, the buffer salts are selected so as not to interfere with the subsequent binding of the metal ion to the chelating agent.

Preferably the radionuclides are readily available from a generator system. For example, ⁶⁸Ga is readily available from a ⁶⁸Ga/⁶⁸Ge generator. Preferably the radiolabelling procedure is fast, the radioactivity incorporation is quantitative (>95%) and the preparation buffer is eligible to human use so that the tracer purification step is omitted. The requirements can be accomplished if the generator eluate is preconcentrated prior to the labelling and the complexation is accelerated by microwave heating.

A method for preconcentration and purification of ⁶⁸Ge/⁶⁸Ga generator eluate was developed (WO 2004/089517; Velikyan I, Beyer G J, Langstrom B. Microwave-supported preparation of ⁶⁸Ga-bioconjugates with high specific radioactivity. Bioconjugate Chem. 2004; 15:554-60). The eluate volume deceases and consequently the concentration of ⁶⁸Ga and macromolecules to be labeled increases. Moreover, the eluate gets purified from the metal cations that might compete with ⁶⁸Ga in the complexation reaction as well as from the long-lived parent ⁶⁸Ge. The method is based on anion exchange chromatography. In HCl solution gallium forms strong anionic complexes with Cl⁻. The corresponding [GaCl₆]³⁻ and [GaCl₄]⁻ complexes are strongly adsorbed at the anion exchange resin from HCl concentrations >3 M and then ⁶⁸Ga is eluted with a small volume of water.

An alternative heating techniques, microwave heating, was applied (WO 2004/089425; Velikyan I, Beyer G J, Langstrom B. Microwave-supported preparation of ⁶⁸Ga-bioconjugates with high-specific radioactivity. Bioconjugate Chem. 2004; 15:554-60); Velikyan I, Lendvai G, Valila M, et al. Microwave accelerated ⁶⁸Ga-labelling of oligonucleotides. J Labelled Compd Rad. 2004; 47:79-89) for the labelling procedures in order to shorten the reaction time and increase the efficiency of the labelling.

A fast method for ⁶⁸Ga-labelling of macromolecules with high specific radioactivity was developed. The method allowed the quantitative incorporation of ⁶⁸Ga, omission of the tracer purification step and a preparation eligible for human use. The method utilized the preconcentrated and purified ⁶⁸Ge/⁶⁸Ga generator eluate, microwave heating and buffers eligible for human use.

The following isotopes or isotope pairs can be used for both imaging and therapy without having to change the radiolabeling methodology or chelator: ⁶⁸Ga and ⁸⁷Y₃₉; ⁶⁸Ga and ¹⁷⁷Lu₇₁; ⁶⁸Ga and ²¹³Bi₈₃.

⁶⁸Ga-based peptide tracers may be used for tumor imaging/diagnosis, chemo- and radiotherapy planning and monitoring as well as pretherapeutical dosimetry for radiotherapy. Furthermore, after the diagnosis and dosimetry ⁶⁸Ga might be substituted in the same vector with a therapeutical radionuclide (⁸⁷Y₃₉, ²¹³_{Bi}₈₃, ¹⁷⁷Lu₇₁) for subsequent radiotherapy. A straightforward preparation of a tracer using radiometallation with generator produced ⁶⁸Ga may result in kit type production of PET radiopharmaceuticals and make PET examinations possible at centres lacking accelerators. Pretherapeutical dosimetry might require accurate quantification, which for some applications is dependent on the specific radioactivity (SRA) of a tracer. This is especially important for the characterisation of high affinity binding sites, such as many peptide receptors. Another factor that necessitates the high SRA is the labelling of highly potent receptor agonists which can induce side effects. It was thus essential to develop a fast and reliable method for ⁶⁸Ga-labelling of various macromolecules with high SRA.

Preferably the radionuclides are readily available from a generator system. For example, ⁶⁸Ga is readily available from a ⁶⁸Ga/⁶⁸Ge generator. Preferably the radiolabelling procedure is fast, the radioactivity incorporation is quantitative (>95%) and the preparation buffer is eligible to human use so that the tracer purification step is omitted. The requirements can be accomplished if the generator eluate is preconcentrated prior to the labelling and the complexation is accelerated by microwave heating.

Microwave heating, providing acceleration of reactions, is an attractive tool for radiolabelling chemistry of short-lived radionuclides. Moreover, during the conventional heating using an oil bath or oven, the walls of the vessel get heated up first, causing a temperature gradient in the solution. Under microwave irradiation the sample is heated from inside more uniformly at each point resulting in very fast heating. Microwave heating is especially useful for microscale organic chemistry, such as radiolabelling where the sample size is comparable to the penetration depth of the microwave field.

The slow radiolabelling kinetics of DOTA-based bifunctional chelators requires elevated temperatures and time. The extensive conventional heating is undesirable because of the potential damage to macromolecules and the relatively short half-life of ⁶⁸Ga. Microwave heating is an attractive tool that can provide acceleration of the labelling.

The microwave heating was applicable without observed degradation of the peptides and oligonucleotides with respective molecular weights of at least up to 7.1 and 9.8 kDa. The pH of the reaction media was adjusted by sodium acetate or N-2-Hydroxyethylpiperazine-N′-2-ethanesulfonic acid buffer (HEPES) as well as sodium hydroxide. The macromolecules, their conjugates and ^{69,71}Ga-comprising counterparts were exposed to microwaves and then analyzed by radio-UV-HPLC and/or LC-ESI-MS to confirm their stability. Compared to synthesis with conventional heating, the application of microwave heating shortened the synthesis time considerably. It should be mentioned that the radiochemical yield of a tracer comprising ⁶⁸Ga radiometal decreases by ˜10% with additional 10 min due to ⁶⁸Ga decay.

But the microwave heating not only reduced the chemical reaction time, it also eliminated side reactions, increased the radioactivity incorporation (RAI), and improved the reproducibility. The ⁶⁸Ga-labelling under microwave heating was performed with small peptides with a molecular weights varying between 1.4-3.3 kDa as well as larger peptides with a molecular weight of 6.2 and 7.1 kDa. A cell and frozen section receptor binding assays were usually performed in order to assess the maintenance of receptor binding capability of tracers.

The theoretical SRA of ⁶⁸Ga is 100 GBq/nmol. The developed labelling technique allowed high SRA values up to 3.3 GBq/nmol considering the generator eluted ⁶⁸Ga radioactivity of 1.25 GBq. This allows a broad range of SRA values and possibility for optimisation of an applied tracer amount in terms of the required mass transport, receptor saturation and image contrast.

The importance of the tracer SRA for the investigation of receptor binding properties was studied by binding saturation of ⁶⁸Ga-DOTATOC to Rhesus monkey brain targeting cortex. The ratios of the local concentrations of ⁶⁸Ga-DOTATOC bound specifically to the receptors, and the free ⁶⁸Ga-DOTATOC reflect the contrast of an image which is critically dependent upon SRA if the amount of radioactivity is kept constant. The bound to free ligand ratio approaches zero when the SRA approaches zero, thus resulting in decreased image contrast. The expression approaches Bₘₐₓ/K_{D} when SRA reaches infinity. At certain level of SRA, B/F reaches the plateau and does not change with increasing SRA. The dependence of the signal-to-background ratio on the SRA is critical around the inflection point. Small decrease in SRA values might bring considerable deterioration of image contrast, and consequently cause irreproducible results. This could be the case when a certain amount of radioactivity is needed to obtain a sufficient signal in vitro, or when the radiactivity dose is the limiting factor as in vivo. In such cases the value of the SRA should be high enough for B/F to lie on the plateau where the signal-to-background ratio becomes independent on variations in SRA. This would provide high reproducibility and robustness of in vivo and in vitro studies, since variations in SRA from one experiment to another would not influence the quantification.

Thus, it has been shown that a sufficiently high SRA might be necessary for receptor quantification, evaluation and sufficient contrast of images. Furthermore, high SRA, achieved by a combination of the preconcentration/purification of ⁶⁸Ga and microwave heating, enables investigation of radioactivity uptake as a function of SRA for optimisation. Moreover, an optimisation of the SRA and binding site quantification might be performed for patient studies when planning the dose for chemo- and radiotherapy. Further improvements of the SRA might also open for a possible determination of Bₘₐₓ in tumours in vivo.

Chemical characterisation and analysis prior to the further application of a radiolabelled compound are necessary to ensure its identity, purity and amount. The analysis should be performed within short time to minimize the loss of radioactivity. For macromolecular bioconjugates, appropriate means of characterisation generally include HPLC and mass spectrometry (MS). The most commonly used method is the addition of the authentic reference substance to the tracer and coelution on an HPLC column connected in series with a radioactivity and a UV detector. This method is convenient and can easily be performed for each synthesis. The HPLC analysis developed in this study is accomplished within 10 min allowing fast quality control (QC) of the peptide-based radiopharmaceutical prior to clinical application. The authentic reference substance was synthesized under the same conditions as its radioactive counterpart, but using a mixture of ⁶⁸Ga and ^{69,71}Ga cations. The aim of the use of a mixture of radioactive and stable gallium isotopes was twofold: 1. to create a reaction condition identical to the labelling procedure; 2. to make it possible to follow the reaction. The identity of the compounds was confirmed by LC-ESI-MS. The position of the ⁶⁸Ga-label was assessed by performing the labelling reaction with both conjugated and nonconjugated macromolecules.

The stability of the radiolabelled bioconjugates both in preparation and application buffers was usually monitored by radio-HPLC with analysis of aliquots taken from the labelling reaction mixture during 3-4 hours to control possible appearance of additional radio-HPLC signals. The samples incubated for 12-24 hours were analyzed by UV-HPLC or LC-ESI-MS. The radiochemical purity of the ⁶⁸Ga-bioconjugates used in the applied studies was >95% for at least four hours. This time corresponds to 3-4 physical half-lives of ⁶⁸Ga and is the time required for the applied experiments. In addition, the macromolecules, bioconjugates and the bioconjugate complexes with the stable gallium isotope were analyzed regarding stability by UV-HPLC or LC-ESI-MS.

To assess the reliability of the designed HPLC system, the quantity of 68Ga-labelled bioconjugate and radio-impurities retained on the column was determined by measuring the radioactivity of the sample injected on the column and the fractions collected from the outlet with a crystal scintillation counter. The overall loss on the system was then estimated and depending on separation methods was 10-15%.

In the present study, the omission of the purification step was possible because of the developed method for quantitative RAI. Moreover, the labelling product was obtained in HEPES buffer which is compatible with biological systems and eligible for human use.

Preferred chelating agents for use in the method of the invention are those which present 68Ga in a physiologically tolerable form. Further preferred chelating agents are those that form complexes with ⁶⁸Ga that are stable for the time needed for diagnostic investigations using the radiolabelled Complexes.

Suitable chelating agents are, for instance, polyaminopolyacid chelating agents like DTPA, EDTA, DTPA-BMA, DOA3, DOTA, HP-DOA3, TMT or DPDP. Those chelating agents are well known for radiopharmaceuticals and radiodiagnosticals. Their use and synthesis are described in, for example, U.S. Pat. No. 4,647,447, U.S. Pat. No. 5,362,475, U.S. Pat. No. 5,534,241, U.S. Pat. No. 5,358,704, U.S. Pat. No. 5,198,208, U.S. Pat. No. 4,963,344, EP-A-230893, EP-A-130934, EP-A-606683, EP-A-438206, EP-A-434345, WO-A-97/00087, WO-A-96/40274, WO-A-96/30377, WO-A-96/28420, WO-A-96/16678, WO-A-96/11023, WO-A-95/32741, WO-A-95/27705, WO-A-95/26754, WO-A-95/28967, WO-A-95/28392, WO-A-95/24225, WO-A-95/17920, WO-A-95/15319, WO-A-95/09848, WO-A-94/27644, WO-A-94/22368, WO-A-94/08624, WO-A-93/16375, WO-A-93/06868, WO-A-92/11232, WO-A-92/09884, WO-A-92/08707, WO-A-91/15467, WO-A-91/10669, WO-A-91/10645, WO-A-91/07191, WO-A-91/05762, WO-A-90/12050, WO-A-90/03804, WO-A-89/00052, WO-A-89/00557, WO-A-88/01178, WO-A-86/02841 and WO-A-86/02005.

Suitable chelating agents include macrocyclic chelating agents, e.g. porphyrin-like molecules and pentaaza-macrocycles as described by Zhang et al., Inorg. Chem. 37(5), 1998, 956-963, phthalocyanines, crown ethers, e.g. nitrogen crown ethers such as the sepulchrates, cryptates etc., hemin (protoporphyrin IX chloride), heme and chelating agents having a square-planar symmetry.

Macrocyclic chelating agents are preferably used in the method of the invention. In a preferred embodiment, these macrocyclic chelating agents comprise at least one hard donor atom such as oxygen and/or nitrogen like in polyaza- and polyoxomacrocycles. Preferred examples of polyazamacrocyclic chelating agents include NOTA, DOTA, TRITA, TETA and HETA with DOTA being particularly preferred.

Particularly preferred macrocyclic chelating agents comprise functional groups such as carboxyl groups or amine groups which are not essential for coordinating to Ga³⁺ and thus may be used to couple other molecules, e.g. targeting vectors, to the chelating agent. Examples of such macrocyclic chelating agents comprising functional groups are NOTA, DOTA, TRITA or HETA.

In a further preferred embodiment, bifunctional chelating agents are used in the method according to the invention. “Bifunctional chelating agent” in the context of the invention mean chelating agents that are linked to a targeting vector comprising RGD peptides.

The targeting vector can be linked to the chelating agent via a linker group or via a spacer molecule. Examples of linker groups are disulfides, ester or amides, examples of spacer molecules are chain-like molecules, e.g. lysin or hexylamine or short peptide-based spacers. In a preferred embodiment, the linkage between the targeting vector and the chelating agent part of radiolabelled gallium complex is as such that the targeting vector can interact with its target in the body without being blocked or hindered by the presence of the radiolabelled gallium complex.

A preferred aspect of the invention is a method for producing a ⁶⁸Ga-radiolabelled complex by
a) obtaining ⁶⁸Ga by contacting the eluate from a ⁶⁸Ge/⁶⁸Ga generator with an anion exchanger comprising HCO₃⁻ as counterions and eluting ⁶⁸Ga from said anion exchanger, and
b) reacting the ⁶⁸Ga with a chelating agent which is conjugated with a targeting vector, wherein the reaction is carried out using microwave heating.

A temperature control under microwave heating of the reaction is advisable when temperature sensitive chelating agents, like for instance bifunctional chelating agents conjugated with peptides or proteins as targeting vectors, are employed in the method according to the invention. Duration of the microwave heating should be adjusted in such a way, that the temperature of the reaction mixture does not lead to the decomposition of the chelating agent and/or the targeting vector. If chelating agents used in the method according to the invention comprise peptides or proteins, higher temperatures applied for a shorter time are generally more favourable than lower temperatures applied for a longer time period.

Microwave heating can be carried out continuously or in several microwave heating cycles during the course of the reaction.

The diagnostic agents of the invention may be administered to patients for imaging in amounts sufficient to yield the desired results with the particular imaging technique.

The compounds according to the invention may therefore be formulated for administration using physiologically acceptable carriers or excipients in a manner fully within the skill of the art. For example, the compounds, optionally with the addition of pharmaceutically acceptable excipients, may be suspended or dissolved in an aqueous medium, with the resulting solution or suspension then being sterilized.

Use of the compounds of formula I in the manufacture of therapeutic compositions (medicament) and in methods of therapeutic or prophylactic treatment, preferably treatment of cancer, of the human or animal body are thus considered to represent further aspects of the invention.

Thus, the present invention relates to a method of producing radiolabelled metal complexes. The complexes could be used as diagnostic and therapeutic agents, e.g. for positron emission tomography (PET), single photon emission computed tomography (SPECT) imaging, pretherapeutical dosimetry, therapy planning, therapy monitoring and radiotherapy.

Viewed from one aspect the invention provides a method of generating an image of a human or animal body involving administering a diagnostic imaging agent to said body, e.g. into the vascular system and generating an image of at least a part of said body to which said diagnostic imaging agent has distributed using scintigraphy, PET or SPECT modalities, wherein as said diagnostic imaging agent is used an agent of formula I.

Viewed from another aspect the invention provides method of monitoring the effect of treatment of a human or animal body with a drug to combat a condition associated with cancer, preferably angiogenesis, e.g. a cytotoxic agent or radiotherapeutics said method involving administering to said body an agent of formula I and detecting the uptake of said agent by cell receptors, preferably endothelial cell receptors and in particular αvβ3 receptors, said administration and detection optionally but preferably being effected repeatedly, e.g. before, during and after treatment with said drug.

Further, it provides a method of pretherapeutical dosimetry to measure the radioactivity amount taken up by tumors and normal organs. This would provide information on how much toxic radioactivity goes to the tumor and if normal organs are affected. It is an easy and quick estimation of dose to tumor and normal tissue. The said method involves administering to a human or animal body an agent of formula I and detecting the uptake of said agent by cell receptors, preferably endothelial cell receptors and in particular αᵥβ₃ receptors, said administration and detection being conducted before the treatment and for planning the treatment.

Still further, it provides a method of therapy planning to determine the appropriate therapy type. This would provide information of receptor density in vivo. The said method involves administering to said body an agent of formula I, quantifying the receptor density in vivo by measuring receptor uptake over expressing tissue and determining appropriate therapy type.

In yet another embodiment of the instant invention, it provides compounds of Formula III, IV and V and methods of using compounds of Formula III, IV and V for radiotherapy. It also provides a method of radiotherapy comprises administering to a human or animal body an agent of compounds of Formula III, IV or V.

In view of the relatively short half-life of ⁶⁸Ga there is a need for a fast method for the synthesis of ⁶⁸Ga-labelled complexes, which could be used as tracer molecules for PET imaging.

The invention thus provides a method of producing a radiolabelled gallium complex by reacting a Ga³⁺ radioisotope with a chelating agent characterised in that the reaction is carried out using microwave heating.

Hence, another preferred embodiment of the method according to the invention is a method of producing a ⁶⁸Ga-radiolabelled complex by reacting ⁶⁸Ga³⁺ with a chelating agent using microwave heating, wherein the ⁶⁸Ga³⁺ is obtained by contacting the eluate form a ⁶⁸Ge/⁶⁸Ga generator with an anion exchanger, preferably with an anion exchanger comprising HCO₃⁻ as counterions, and eluting ⁶⁸Ga³⁺ from said anion exchanger.

The peptide portion of the compounds of the present invention can be synthesised using all the known methods of chemical synthesis but particularly useful is the solid-phase methodology of Merrifield employing an automated peptide synthesiser (J. Am. Chem. Soc., 85: 2149 (1964)). The peptides and peptide chelates may be purified using high performance liquid chromatography (HPLC) and characterised by mass spectrometry and analytical HPLC before testing in the in vitro screen.

The present invention will now be further illustrated by way of the following non-limiting examples.

### Examples

### ⁶⁸Ga-Labelling of Cys2-6; c[CH₂CO-Lys(DOTA)-Cys-Arg-Glv-Asp-Cvs-Phe-Cys]-CCX₆—NH₂) (DOTA-AH-110847-02)

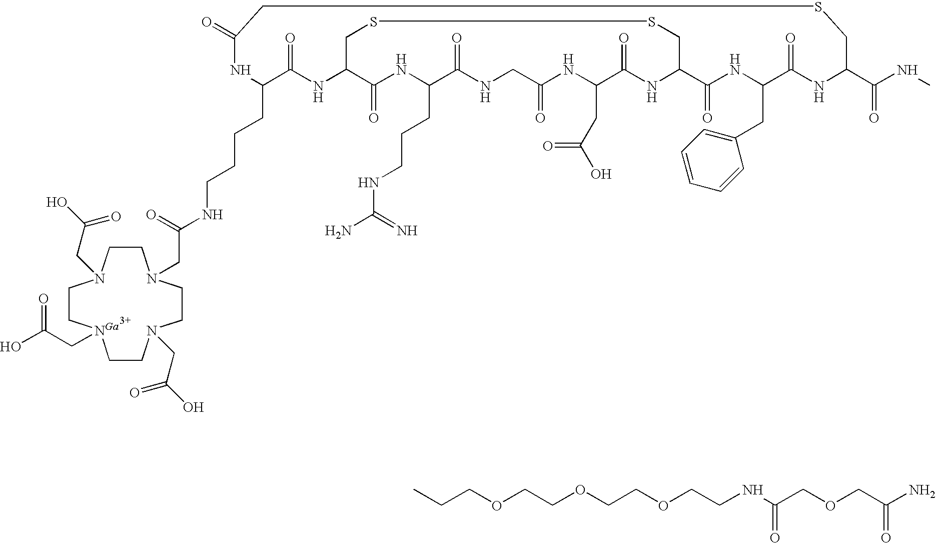

Materials

DOTA-AH-110847-02 (C₆₆H₁₀₅N₁₉O₂₄S₃, Molecular Weight=1644.88) was received from Amersham Health (Dept. of Synthetic Chemistry, Amersham Health, Oslo, Norway). HEPES (4-(2-Hydroxyethyl)piperazine-1-ethanesulfonic acid) and double distilled hydrochloric acid (Riedel de Haën) were obtained from Sigma-Aldrich Sweden (Stockholm, Sweden). Sodium dihydrogen phosphate, disodium hydrogen phosphate and trifluoroacetic acid (TFA) were obtained from Merck (Darmstadt, Germany). The purchased chemicals were used without further purification. Deionised water (18.2 MΩ), produced with a Purelab Maxima Elga system (Bucks, UK) was used in all reactions.

⁶⁸Ga Production

⁶⁸Ga (T_{1/2}=68 min, β⁺=89% and EC=11%) was available from a ⁶⁸Ge/⁶⁸Ga-generator-system (Cyclotron Co., Ltd, Obninsk, Russia) where the ⁶⁸Ge (T_{1/2}=270.8 d) was attached to a column of an inorganic matrix based on titanium dioxide. The nominal ⁶⁸Ge activity loaded onto the generator column was 1850 MBq (50 mCi). The specified shelf-live of the generator is 2-3 years. The ⁶⁸Ga was eluted with 6 mL of 0.1 M hydrochloric acid.

Purification and Preconcentration of the ⁶⁸Ga Eluate Using an Anion Exchange Cartridge

The ⁶⁸Ge/⁶⁸Ga-generator was eluted according to the manufacturer protocol with 6 mL 0.1 M solution. 5 mL of 30% HCl was added to the 6 mL of the generator eluate giving finally a HCl concentration of 4.0 M. The resulting 11 mL solution in total was passed through an anion exchange column at a flow rate of 4 mL/min (linear flow speed 25 cm/min) at room temperature. The ⁶⁸Ga was then eluted with small fractions of deionized water (50-200 μl) at a flow rate of 0.5 mL/min.

The pre-concentration has successfully been performed for the eluates (12 mL) of two generators. This means that the useful shelf-life of the generators can be even longer.

⁶⁸Ga-Labelling of DOTA-AH-110847-02

The pH of the pre-concentrated/purified ⁶⁸Ge/⁶⁸Ga-generator eluates was adjusted to pH 4.6-4.8 by adding sodium hydroxide and HEPES (4-(2-Hydroxyethyl) piperazine-1-ethanesulfonic acid, Sigma) to give finally a 1.5 M solution with regard to HEPES. Then 3-8 nanomols of the peptide conjugates were added. The reaction mixture was transferred to a Pyrex glass vial with an insert to accommodate the small volume (200±20 μL) for microwave heating. The heating time in the microwave oven was 1 min at 95±5° C. The obtained product was analyzed by UV-radio-HPLC using reverse phase separation mechanism.

Microwave-Heating

The microwave heating was performed in a SmithCreator™ monomodal microwave cavity producing continuous irradiation at 2450 MHz (former Personal Chemistry AB, now Biotage, Uppsala, Sweden). The temperature, pressure and irradiation power were monitored during the course of the reaction. The reaction vial was cooled down with pressurized air after completed irradiation.

HPLC Analysis

Analytical liquid chromatography (LC) was performed using a HPLC system from Beckman (Fullerton, Calif., USA) consisting of a 126 pump, a 166 UV detector and a radiation detector coupled in series. Data acquisition and handling was performed using the Beckman System Gold Nouveau Chromatography Software Package. The column used was a Vydac RP 300 Å HPLC column (Vydac, USA) with the dimensions 150 mm×4.6 mm, 5 μm particle size. The gradient elution was applied with the following parameters: A=10 mM TFA; B=70% acetonitrile (MeCN), 30% H₂O, 10 mM TFA with UV-detection at 220 nm; flow was 1.2 mL/min; 0-2 min isocratic 20% B, 20-90% B linear gradient 8 min, 90-20% B linear gradient 2 min.

LC-ESI-MS Analysis

Liquid chromatography electrospray ionization mass spectrometry (LC-ESI-MS) was performed using a Fisons Platform (Micromass, Manchester, UK) with positive mode scanning and detecting [M+2H]²⁺ and [M+3H]³⁺ species. DOTA-AH-110847-02 at m/z=549.1 for [M+2H]²⁺ and 823.44 for [M+3H]³⁺ and ^{69,71}Ga-DOTA-AH-110847-02 at m/z=857 for [M+2H]²⁺ and 571 for [M+3H]³⁺. The ^{69,71}Ga-conjugate synthesised under identical to labeling conditions was used for the identification of the radio-HPLC chromatogram signals.

Radiochemistry

The bicyclic-octapeptide, DOTA-AH-110847-02, conjugated with a macrocyclic bifunctional chelator (DOTA) at Lysine residue has been labelled with positron emitting radionuclide ⁶⁸Ga. The full ⁶⁸Ga radioactivity eluted from two generators was quantitatively (>95%) incorporated into 3-8 nanomols of the peptide conjugate. Further purification of the ⁶⁸Ga labeled peptide conjugate was not required since the nuclide incorporation was quantitative and the buffer was compatible with the biological systems. The over-all ⁶⁸Ga-labelling process was performed in 15-20 min starting from the end of the original generator elution. The HPLC quality control (another 10 minutes) was performed prior to the application.

The original DOTA-AH-110847-02 peptide conjugate was analyzed by UV-RP-HPLC (t_{R}=6.19±0.03).

The labelling of DOTA-AH-110847-02 with ⁶⁸Ga (Scheme 1) was carried out in a microwave oven.

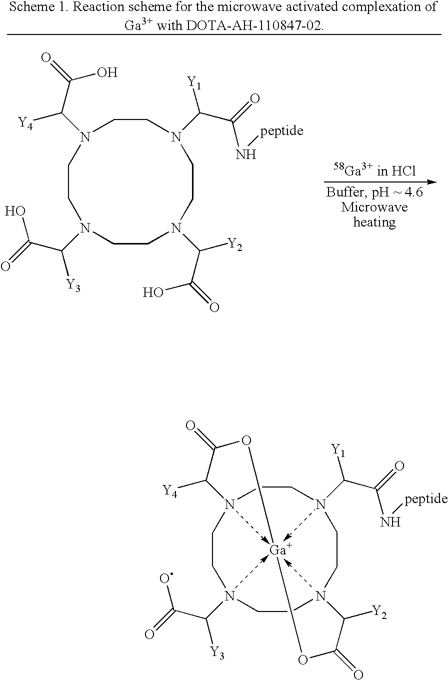

The radioactivity incorporation was >95%. The UV-radio-HPLC analysis method developed for this study was accomplished within 10 min allowing fast quality control (QC) of the radiopharmaceutical prior to the application. Authentic reference substance was synthesized under the same conditions as its radioactive counterpart, but using the stable ^{69,71}Ga isotopes. The retention times (UV-HPLC) were 6.19 and 6.35 respectively for the authentic DOTA-AH-110847-02 and ^{69,71}Ga-DOTA-AH-110847-02. The radioactivity signal had t_{R}=6.45. To confirm the HPLC UV-chromatogram signals DOTA-AH-110847-02 and ^{69,71}Ga-DOTA-AH-110847-02 concentration dependent studies were carried out. The area of the corresponding signals was increasing with the increasing concentration of the analytes. The identity of the compounds was further confirmed by LC-ESI-MS. Double and triple charged ions at m/z: 857 [M+2H]²⁺ and 571 [M+3H]³⁺ were detected for ^{69,71}Ga-AH-110847-02. In the case of the gallium stable isotope complexation, the product consisted of pure ^{69,71}Ga-DOTA-AH-110847-02. In the case of ⁶⁸Ga complexation, the product consists of ⁶⁸Ga-DOTA-AH-110847-02 and DOTA-AH-110847-02. The HPLC samples of the tracer preparations were spiked with DOTA-AH-110847-02 to avoid t_{R} discrepancies.

To check the stability of DOTA-RDG under microwave heating condition, blank experiments were performed and the stability of the peptide conjugate was monitored by UV-HPLC and LC-ESI-MS. DOTA-RDG was stable both in water and in the reaction solution under MW-irradiation. No additional signals were detected in the stability study.

### Specific Embodiments, Citation of References

The present invention is not to be limited in scope by specific embodiments described herein. Indeed, various modifications of the inventions in addition to those described herein will become apparent to these skilled in the art from the foregoing description and accompanying figures. Such modifications are intended to fall within the scope of the appended claims.

Various publications and patent applications are cited herein, the disclosures of which are incorporated by reference in their entireties.

What is claimed is:

1. A compound of general formula (I) 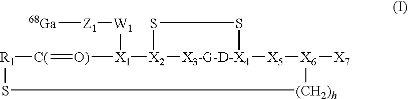
or pharmaceutically acceptable salt thereof
wherein
G represents glycine
D represents aspartic acid
R₁ represents —(CH₂)ₙ— or —(CH₂)ₙ—C₆H₄— wherein
n represents a positive integer 1 to 10
h represents a positive integer 1 or 2
X₁ represents an amino acid residue wherein said amino acid possesses a functional side-chain such as an acid or amine.
X₂ and X₄ represent independently an amino acid residue capable of forming a disulphide bond,
X₃ represents arginine, N-methylarginine or an arginine mimetic,
X₅ represents a hydrophobic amino acid or derivatives thereof, and
X₆ represents a thiol-containing amino acid residue, and
X₇ is absent or represents a biomodifier moiety
Z₁ represents a chelating agent.

2. A compound as claimed in claim 1 wherein any of the amino acid residues are independently in the D or L conformation.

3. A compound as claimed in claim 1 wherein R₁ represents —(CH₂)—.

4. A compound as claimed in claim 1 wherein X₁ represents aspartic acid, glutamic acid,lysine, homolysine or a diaminoalkylic acid or derivatives thereof.

5. A compound as claimed in claim 1 wherein X₂, X₄ and X₆ independently represent a cysteine or homocysteine residue.

6. A compound as claimed in claim 1 wherein X₃ represents an arginine residue.

7. Compound as claimed in claim 1 wherein X₅ represents a tyrosine, a phenylalanine, a 3-iodo-tyrosine or a naphthylalanine residue.

8. A compound as claimed in claim 1 wherein X₇ is absent or comprises 1-10 units of a monodisperse PEG building block.

9. A compound as claimed in claim 1 wherein X₇ is absent or comprises 1-10 units of Formula II

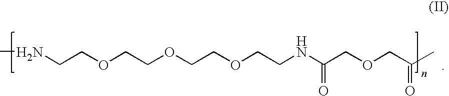

10. A compound as claimed in claim 1 wherein X₇ represent 1-10 amino acid residues

11. A compound as claimed in claim 1 wherein X₇ represent glycine, lysine, aspartic acid or serine residues, preferably glycine.

12. A compound as claimed in claim 1 where Z₁ is NOTA, DOTA or TETA.

13. A compound as claimed in claim 1 where Z₁ is an antineoplastic agent.

14. A compound as claimed in claim 13 where Z₁ represent cyclophosphamide, chloroambucil, busulphan, methotrexate, cytarabine, fluorouracil, vinblastine, paclitaxel, doxorubicin, daunorubicin, etoposide, teniposide, cisplatin, amsacrine or docetaxel.

15. A compound as claimed in claim 1 where W₁ is glutaric or succinic acid

16. A compound as claimed in claim 1 defined by the following formula Compound I

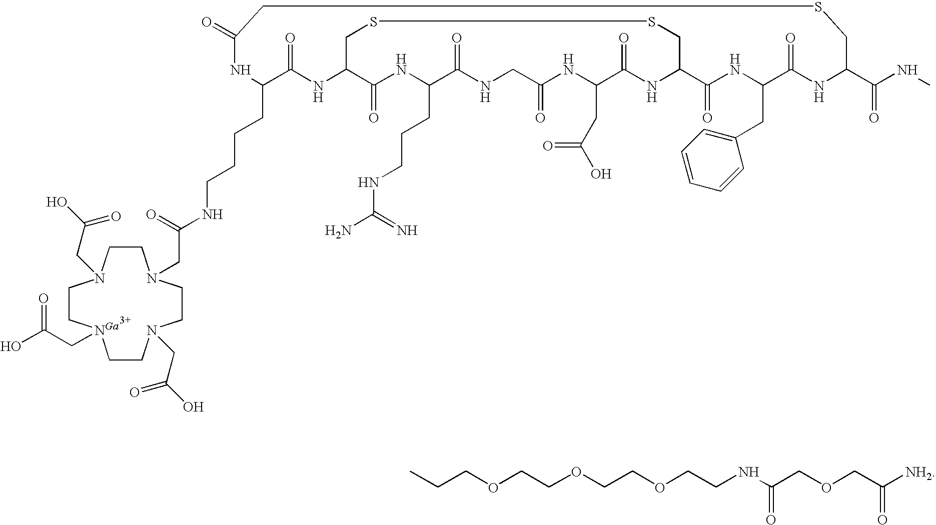

17. A pharmaceutical composition comprising an effective amount of a compound of general Formula (I) or a salt thereof, together with one or more pharmaceutically acceptable adjuvants, excipients or diluents for use in enhancing image results in in vivo imaging.

18. Use of a compound as claimed claim 1 for the manufacture of a diagnostic imaging agent for use in a method of diagnosis involving administering said diagnostic imaging agent to a human or animal body and generating an image of at least part of said body.

19. A method of generating images of a human or animal body involving administering a diagnostic imaging agent to said body, and generating an image of at least a part of said body to which said diagnostic imaging agent has distributed, characterised in that said diagnostic imaging agent comprises a compound as claimed in claim 1.

20. A method of pretherapeutical dosimetry comprising administering to a human or animal body a compound of formula I, or an acid addition salt thereof, together with one or more pharmaceutically acceptable adjuvants, excipients or diluents, detecting the uptake of said compound by cell receptors, preferably endothelial cell receptors and in particular αᵥβ₃ receptors, said administration and detection being conducted before the treatment and for the planning of the treatment.

21. A method of monitoring the effect of treatment of a human and animal body with a drug to combat a condition associated with cancer, preferably angiogenesis, comprising administering to said body a compound of formula I, or an acid addition salt thereof, together with one or more pharmaceutically acceptable adjuvants, excipients or diluents, detecting the uptake of said compound by cell receptors, preferably endothelial cell receptors and in particular αᵥβ₃receptors, said administration and detection being conducted repeatedly before, during and after treatment with said drug.

22. A method of therapy planning to determine the appropriate therapy type to a human and animal body comprising administering to said body a compound of formula I, or an acid addition salt thereof, together with one or more pharmaceutically acceptable adjuvants, excipients or diluents, quantifying receptor density in vivo by measuring receptor uptake over expressing tissue and determining appropriate therapy type.

23. A compound of general formula (III), (IV) or (V) 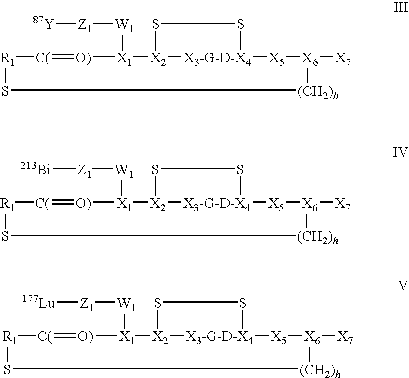
or pharmaceutically acceptable salt thereof
wherein
G represents glycine
D represents aspartic acid
R₁ represents —(CH₂)ₙ— or —(CH₂)ₙ—C₆H₄— wherein
n represents a positive integer 1 to 10
h represents a positive integer 1 or 2
X₁ represents an amino acid residue wherein said amino acid possesses a functional side-chain such as an acid or amine.
X₂ and X₄ represent independently an amino acid residue capable of forming a disulphide bond,
X₃ represents arginine, N-methylarginine or an arginine mimetic,
X₅ represents a hydrophobic amino acid or derivatives thereof, and
X₆ represents a thiol-containing amino acid residue, and
X₇ is absent or represents a biomodifier moiety
Z₁ represents a chelating agent.

24. A method of radiotherapy of cancer, preferably angiogenesis, in a human or animal body, comprising the administering of an effective amount of a compound III, IV or V.

25. Use of a compound as claimed in claim 23 for the manufacture of a medicament for the radiotherapy treatment of cancer, preferably angiogenesis, in a human or animal.
